(19) United States
(12) Patent Application Publication
(10) Pub. No.: US 2018/0104234 A1
(43) Pub. Date: Apr. 19, 2018

(54) COMBINATION THERAPY FOR CANCER

(71) Applicant: Eli Lilly and Company, Indianapolis, IN (US)

(72) Inventors: Gregory Paul Donoho, Indianapolis, IN (US); Volker Wacheck, Vienna (AT)

(21) Appl. No.: 15/562,541

(22) PCT Filed: May 4, 2016
(86) PCT No.: PCT/US2016/030697

§ 371 Date: Sep. 28, 2017

Related U.S. Application Data
(60) Provisional application No. 62/158,694, filed on May 8, 2015.

(51) Int. Cl. A61K 31/4745; A61P 35/00; C07K 16/28; A61K 39/395; A61K 9/00

(52) U.S. Cl. CPC A61K 31/4745; A61P 35/00; C07K 16/2863; A61K 2039/54; A61K 9/0053; A61K 9/0019; C07K 2317/21; A61K 39/3955

(57) ABSTRACT

The present invention provides a method of treating squamous histology cancers and PI3K pathway activated large cell lung and colorectal cancers in a patient comprising administering to a patient in need of such treatment an effective amount of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof; in combination with an effective amount of necitumumab.

The present invention relates to a combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one with necitumumab and to methods of using the combination to treat certain disorders, such as cancer, in particular squamous histology cancers and PI3K pathway activated large cell lung and colorectal cancers including, most particularly, squamous non-small cell lung cancer (squamous NSCLC).

The present invention is in the field of treatment of squamous histology cancers and phosphoinositide 3 kinase (PI3K) pathway activated large cell lung and colorectal cancers including squamous NSCLC, head and neck squamous cell carcinoma (HNSCC), squamous anal cancer, squamous bladder cancer, and squamous thyroid cancer as well as non-squamous large cell lung cancer and non-squamous colorectal cancer (CRC). These types of cancer have a significant impact on numerous patients. For example, the subtypes of NSCLC include 25% to 30% that are squamous cell carcinoma and 10% that are large cell carcinoma. Furthermore, HNSCC represents the sixth leading cancer by incidence and there are approximately 500,000 new cases a year worldwide.

Unfortunately, broadly applicable therapies for squamous histology cancers and PI3K pathway activated large cell lung and colorectal cancers still remains elusive and, thus, there exists a need for more and different therapies that may prove to be effective in treating squamous histology cancers and PI3K pathway activated large cell lung and colorectal cancers.

8-[5-(1-hydroxy-1-methylethyl)pyridin-3-yl]-1-[(2S)-2-methoxypropyl]-3-methyl-1,3-dihydro-2H-imidazo[4,5-c]quinolin-2-one is a dual inhibitor of PI3K and mTOR (mammalian target of rapamycin) kinase. The compound and methods of making and using this compound including for the treatment of cancer and more specifically for the treatment of NSCLC are disclosed in WO 2012/097039. Furthermore, a clinical investigation is ongoing for the compound in patients with squamous NSCLC.

Necitumumab is a recombinant IgG1 human monoclonal antibody targeting the epidermal growth factor receptor (EGFR). Necitumumab and methods of making and using this antibody including for the treatment of neoplastic diseases such as solid and non-solid tumors are disclosed in U.S. Pat. No. 7,598,350. Furthermore, clinical activity for necitumumab has also been reported in patients with NSCLC (Thatcher, N., et al. J Clin Oncol 32.5 Suppl (2014), (ASCO presentation 2014) http://meetinglibrary.asco.ong/content/125543-144 (discussing “A randomized, multicenter, open-label, phase 111 study of gemcitabine-cisplatin (GC) chemotherapy plus necitumumab (IMC-11F8/LY3012211) versus GC alone in the first-line treatment of patients (pts) with stage IV squamous non-small cell lung cancer (sq-NSCLC).”).

Novel methods of using the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab to treat squamous histology cancers and PI3K pathway activated large cell lung and colorectal cancers is herein presented. Combinations of PI3 kinase/mTOR inhibitors and EGFR inhibitors have been contemplated in the art. More particularly, disclosures include certain combinations of cetuximab, an EGFR inhibitor, with PKI-587, a dual PI3K/mTOR inhibitor, in EGFR-resistant human head and neck cancer models (Amato et al., BJC (2014) 110, 2887-2895) and of cetuximab with BYL719, a selective α-isoform PI3K inhibitor, in preclinical and clinical settings for squamous cell carcinoma of the head and neck (Munster et al., Poster A46, Targeting the PI3K-mTOR Network in Cancer AACR Special Conference (Sep. 14-17, 2014; Philadelphia, Pa., USA)). However, the present invention discloses herein methods of treating squamous histology cancers and PI3K pathway activated large cell lung and colorectal cancers that provides enhanced and/or unexpected beneficial therapeutic effects from the combined activity of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab in squamous histology cancers and PI3K pathway activated large cell lung and colorectal cancers patients as compared to the therapeutic effects proved by either agent alone. Furthermore the present invention discloses methods of treating squamous histology cancers and PI3K pathway activated large cell lung and colorectal cancers as part of a specific treatment regimen that provides enhanced and/or unexpected beneficial therapeutic effects from the combined activity of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab in squamous histology cancers and PI3K pathway activated large cell lung and colorectal cancers patients as compared to the therapeutic effects proved by either agent alone.

Accordingly, the present invention provides a method of treating squamous histology cancers and PI3K pathway activated large cell lung and colorectal cancers in a patient, comprising administering to a patient in need of such treatment an effective amount of a compound of the formula:

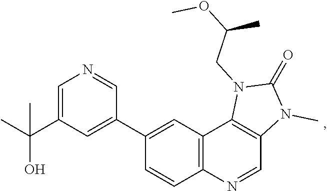 [SMILES: CO[C@@H](C)Cn1c(=O)n(C)c2cnc3ccc(-c4cncc(C(C)(C)O)c4)cc3c21]

or a pharmaceutically acceptable salt thereof, in combination with an effective amount of necitumumab. A compound of the formula:

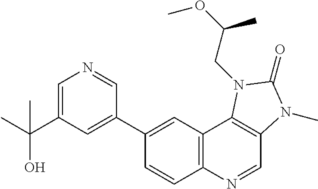 [SMILES: CO[C@@H](C)Cn1c(=O)n(C)c2cnc3ccc(-c4cncc(C(C)(C)O)c4)cc3c21]

refers to the compound named 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The present invention also provides a method of treating squamous histology cancers and PI3K pathway activated large cell lung and colorectal cancers in a patient, comprising administering to a patient in need of such treatment an effective amount of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, in combination with an effective amount of necitumumab wherein the compound or salt thereof is administered at a dose of about 200 mg twice per day of a 21-day cycle and necitumumab is administered at a dose of about 800 mg on Days 1 and 8 of a 21-day cycle. Preferably, the compound or salt thereof is administered orally and necitumumab is administered intravenously.

In addition, the invention provides a kit comprising a compound of the formula:

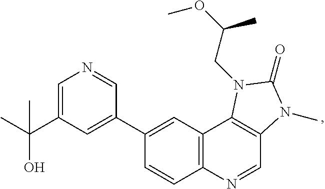 [SMILES: CO[C@@H](C)Cn1c(=O)n(C)c2cnc3ccc(-c4cncc(C(C)(C)O)c4)cc3c21]

or a pharmaceutically acceptable salt thereof, and necitumumab for the treatment of squamous histology cancers and PI3K pathway activated large cell lung and colorectal cancers. A compound of the formula:

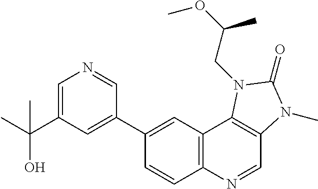 [SMILES: CO[C@@H](C)Cn1c(=O)n(C)c2cnc3ccc(-c4cncc(C(C)(C)O)c4)cc3c21]

refers to the compound named 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The invention further provides a kit, comprising a pharmaceutical composition, comprising a compound of the formula:

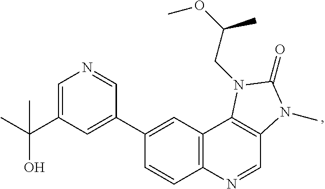 [SMILES: CO[C@@H](C)Cn1c(=O)n(C)c2cnc3ccc(-c4cncc(C(C)(C)O)c4)cc3c21]

or a pharmaceutically acceptable salt thereof, with one or more pharmaceutically acceptable carriers, diluents, or excipients, and a pharmaceutical composition, comprising necitumumab, with one or more pharmaceutically acceptable carriers, diluents, or excipients for the treatment of squamous histology cancers and PI3K pathway activated large cell lung and colorectal cancers. A compound of the formula:

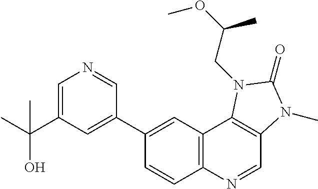 [SMILES: CO[C@@H](C)Cn1c(=O)n(C)c2cnc3ccc(-c4cncc(C(C)(C)O)c4)cc3c21]

refers to the compound named 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The invention further provides a combination comprising a compound of the formula:

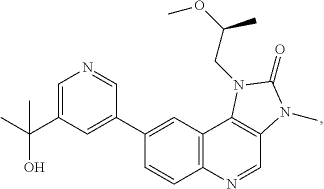 [SMILES: CO[C@@H](C)Cn1c(=O)n(C)c2cnc3ccc(-c4cncc(C(C)(C)O)c4)cc3c21]

or a pharmaceutically acceptable salt thereof and necitumumab, for simultaneous, separate or sequential use in the treatment of squamous histology cancers and PI3K pathway activated large cell lung and colorectal cancers. A compound of the formula:

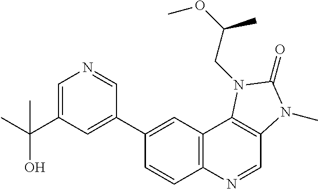 [SMILES: CO[C@@H](C)Cn1c(=O)n(C)c2cnc3ccc(-c4cncc(C(C)(C)O)c4)cc3c21]

refers to the compound named 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The invention further provides a combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and necitumumab for simultaneous, separate or sequential use in therapy. The invention further provides a combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and necitumumab for the manufacture of a medicament for simultaneous, separate or sequential use in the treatment of squamous histology cancers and PI3K pathway activated cancers. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The invention further provides a compound of the formula:

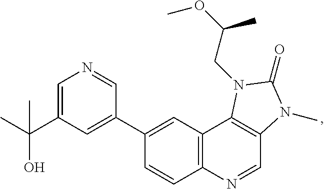 [SMILES: CO[C@@H](C)Cn1c(=O)n(C)c2cnc3ccc(-c4cncc(C(C)(C)O)c4)cc3c21]

or a pharmaceutically acceptable salt thereof, in combination with necitumumab for simultaneous, separate or sequential use in the treatment of squamous histology cancers and PI3K pathway activated large cell lung and colorectal cancers wherein the compound or salt thereof is administered at a dose of about 200 mg twice per day of a 21-day cycle and necitumumab is administered at a dose of about 800 mg on Days 1 and 8 of a 21-day cycle. A compound of the formula:

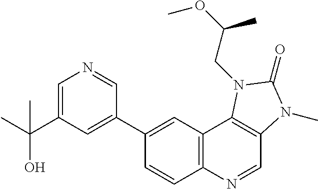 [SMILES: CO[C@@H](C)Cn1c(=O)n(C)c2cnc3ccc(-c4cncc(C(C)(C)O)c4)cc3c21]

refers to the compound named 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

The invention also provides a compound of the formula 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, in combination with necitumumab for simultaneous, separate or sequential use in the treatment of squamous histology cancers and PI3K pathway activated large cell lung and colorectal cancers. More particularly, these squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC. Yet more particularly, the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC. Even more particularly, the squamous histology cancers and PI3K pathway activated large cell lung and colorectal cancers are squamous NSCLC, HNSCC, squamous bladder cancer, and squamous thyroid cancer, and, most particularly, is squamous NSCLC.

As used herein, the compound's name “8-[5-(1-hydroxy-1-methylethyl)pyridin-3-yl]-1-[(2S)-2-methoxypropyl]-3-methyl-1,3-dihydro-2H-imidazo[4,5-c]quinolin-2-one” is disclosed in WO 2012/097039 and refers to the compound with the following structure:

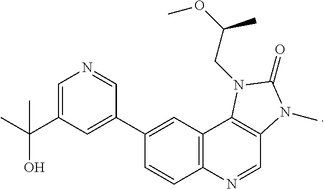 [SMILES: CO[C@@H](C)Cn1c(=O)n(C)c2cnc3ccc(-c4cncc(C(C)(C)O)c4)cc3c21]

This compound's CAS registry number is 1386874-06-1. Alternative compound names include 2H-Imidazo[4,5-c]quinolin-2-one, 1,3-dihydro-8-[5-(1-hydroxy-1-methylethyl)-3-pyridinyl]-1-[(2S)-2-methoxypropyl]-3-methyl-.

“Epidermal growth factor receptor” or “EGFR” is a member of the ErbB (erythroblastic leukemia viral oncogene homolog) family of receptor tyrosine kinases. EGFR activation occurs in response to ligand stimulation and/or genetic alterations of the EGFR gene, such as somatic mutations, amplifications, or deletions. Activated EGFR induces downstream signaling through the MAPK (mitogen-activated protein kinases), PI3K/AKT (phosphoinositide 3-kinase/v-Akt murine thymoma viral oncogene), and PLCγ (phospholipase Cγ) signal transduction pathways that mediate cell proliferation, cell survival, and cell migration, respectively, thereby contributing to neoplastic transformation and tumor growth.

Necitumumab is a recombinant IgG1 human monoclonal antibody designed to bind and block the ligand binding site of EGFR. As used herein, the term “necitumumab” is also known as IMC-11F8, CAS registry number 906805-06-9. The invention provides for necitumumab in various aspects disclosed herein. Necitumumab is an antibody specific for human EGFR and comprises a heavy chain variable region (VH) having the amino acid sequence: QVQLQESGPGLVKPSQTLSLTCTVSGGSISSGDYYWSWIRQPPGKGLEWIGYIYY SGSTDYNPSLKSRVTMSVDTSKNQFSLKVNSVTAADTAVYYCARVSIFGVGTFD YWGQGTLVTVSS (SEQ NO:1) and a light chain variable region (VL) having the amino acid sequence: EIVMTQSPATILSLSPGERATLSCRASQSVSSYLAWYQQKPGQAPRLLIYDASNRA TGIPARFSGSGSGTDFILTISSLEPEDFAVYYCHQYGSTPLTFGGGTKAEIK (SEQ ID NO:2). U.S. Pat. No. 7,598,350.

As used herein, the term “kit” refers to a package comprising at least two separate containers, wherein a first container contains 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and a second container contains necitumumab. A “kit” may also include instructions to administer all or a portion of the contents of these first and second containers to a cancer patient, preferably a squamous NSCLC, HNSCC, squamous anal cancer, squamous bladder cancer, squamous thyroid cancer, non-squamous large cell lung cancer, and non-squamous CRC patient, and more preferably a squamous NSCLC patient.

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, squamous NSCLC, HNSCC, squamous anal cancer, squamous bladder cancer, squamous thyroid cancer, non-squamous large cell lung cancer, and non-squamous CRC.

A main advantage of the combination treatments of the invention is the ability of producing marked anti-cancer effects in a patient without causing significant toxicities or adverse effects, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, duration of survival, progression free survival, overall response rate, duration of response, and quality of life. The therapeutic agents used in the invention may cause inhibition of metastatic spread without shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and measurement of response through radiological imaging.

As used herein, the term “progressive disease” refers to at least a 20% increase in the sum of the diameters of target lesions, taking as reference the smallest (nadir) sum since the treatment started, or the appearance of one or more new lesions. Requires not only 20% increase, but absolute increase of a minimum of 5 mm over sum.

As used herein, the term “stable disease” refers to neither sufficient shrinkage to qualify for PR nor sufficient increase to qualify for PD, taking as reference the smallest (nadir) sum of diameters since the treatment started.

As used herein, the term “partial response”, refers to a t least a 30% decrease in the sum of diameters of target lesions, taking as reference the baseline sum diameters.

As used herein, the term “objective response” refers to a measurable response, including complete response (CR) or partial response.

As used herein, the term “complete response” or “CR” refers to the disappearance of all target lesions.

As used herein, the term “overall response rate” or “ORR” is based on each patient's best objective response will be determined for all patients evaluable via the Response Evaluation Criteria in Solid Tumors (RECIST) v 1.1 criteria. The ORR (%) will be calculated as the number of patients with best objective response of CR or PR divided by the number of patients with measurable disease at baseline. The best objective response for a given patient will be based on objective responses determined from data obtained up to: progression, the last evaluable assessment in the absence of progression, or initiation of subsequent anticancer therapy. Patients for whom an objective response cannot be determined or for who the best objective response is NE will be considered non-responders. The ORR will be summarized along with the 95% Clopper Pearson confidence interval.

As used herein, the term “time to progression” or “TTP” refers to the time, generally measured in weeks or months, from the time of initial treatment, until the cancer progresses or worsens. Such progression can be evaluated by the skilled clinician. As used herein, the term “progression-free survival” or “PFS” is defined as the time from enrollment until the date of disease progression per RECIST or death by any cause. Patients who have not progressed or died at the time of assessment will be censored at the time of the last date of tumor assessment. Patients who are enrolled but do not receive treatment and patients who have no evaluable visits will be censored on day 1. Patients who received new anti-cancer therapy before disease progression or death will be censored to the last tumor assessment date prior to the new anti-cancer therapy. PP'S will also be analyzed by including the clinical progression date as sensitivity analysis.

As used herein, the term “overall survival” or “OS” is defined as the time from enrollment until the date of death by any cause. Patients who lost follow up or did not die at the time of assessment will be censored at the time of the last known alive date

As used herein, the term “survival” refers to the patient remaining alive, and includes overall survival as well as progression free survival.

As used herein, the term “primary tumor” or “primary cancer” is meant the original cancer and not a metastatic lesion located in another tissue, organ, or location in the subject's body.

As used herein, the term “effective amount” refers to the amount or dose of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and to the amount or dose of necitumumab which, upon single or multiple dose administration to the patient, provides an effective response in the patient under diagnosis or treatment. It is also understood that a combination therapy of the present invention is carried out by, administering 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, together with necitumumab in any manner which provides effective levels of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one or a pharmaceutically acceptable salt thereof, and necitumumab in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, the term “effective response” of a patient or a patient's “responsiveness” to treatment with a combination of agents and similar wording refers to the clinical or therapeutic benefit imparted to a patient upon co-administration of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and necitumumab. Such benefit includes any one or more of: extending survival (including overall survival and progression free survival); resulting in an objective response (including a complete response or a partial response); or improving signs or symptoms of cancer, etc.

8-[5-(1-hydroxy-1-methylethyl)pyridin-3-yl]-1-[(2S)-2-methoxypropyl]-3-methyl-1,3-dihydro-2H-imidazo[4,5-e]quinolin-2-one, or a pharmaceutically acceptable salt thereof, is generally effective over a wide dosage range in the combination of the present invention. For example, dosages per day normally fall within the range of about 50 mg to about 300 mg twice per day, preferably about 100 mg to about 200 ing twice per day, more preferably about 150 mg to about 200 mg twice per day, and most preferably about 200 mg twice per day. In addition, necitumumab, is generally effective over a wide dosage range in the combination of the present invention. For example, dosages per 21-day cycle normally fall within the range of about 400 to 1000 mg with dosages on 2 or 3 days of that cycle, alternatively with dosages one time a week or once every two weeks, preferably about 400 to 1000 mg on Day 1, Day 8, and Day 15 of each 21-day cycle, more preferably about 600 to 900 mg on Day 1 and Day 8 of each 21-day cycle, and most preferably about 800 mg on Day 1 and Day 8 of each 21-day cycle. In some instances dosage levels below the lower limit of the aforesaid ranges for 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and necitumumab, may be more than adequate, while in other cases smaller or still larger doses may be acceptably employed, and therefore the above dosage range is not intended to limit the scope of the invention in any way. When given in combination with necitumumab, for example, over a 21-day cycle, 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one or a pharmaceutically acceptable salt thereof is administered daily within the range of 50 mg to about 300 mg twice per day, preferably about 100 mg to about 200 mg twice per day, more preferably about 150 mg to about 200 mg twice per day, and most preferably about 200 mg twice per day of a 21-day cycle and necitumumab is administered within the range of 400 to 1000 trig with dosages on 2 or 3 days of a 21-day cycle, alternatively with dosages one time a week or once every two weeks, preferably about 400 to 1000 mg on Day 1, Day 8, and Day 15 of a 21-day cycle, more preferably about 600 to 900 mg on Day 1 and Day 8 of a 21-day cycle, and most preferably about 800 mg on Day 1 and Day 8 of a 21-day cycle. Additional 21-day cycles can be utilized as needed for treatment of the patient in need thereof.

The free base compound, 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, is preferred. However, it will be understood by the skilled reader that 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one is capable of forming salts. 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2002); L. D. Bighley, S. M. Berge, D. C. Monkhouse, in “Encyclopedia of Pharmaceutical Technology’. Eds. J. Swarbrick and J. C. Boylan, Vol. 13, Marcel Dekker, Inc., New York, Basel, Hong Kong 1995, pp. 453-499; S. M. Berge, et al., “Pharmaceutical Salts”, Journal of Pharmaceutical Sciences, Vol 66, No. 1, January 1977.

8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, and necitumumab are preferably formulated as pharmaceutical compositions administered by any route which makes the compound bioavailable. The route of administration may be varied in any way, limited by the physical properties of the drugs and the convenience of the patient and the caregiver. Preferably, 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, is administered orally. Alternatively, 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, is formulated for parenteral administration, such as intravenous or subcutaneous administration. Preferably, necitumumab is formulated for parenteral administration, such as intravenous or subcutaneous administration, more preferably for intravenous administration. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006).

As used herein, the phrase “in combination with” refers to the administration of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof; with necitumumab simultaneously. As used herein, the phrase “in combination with” also refers to the administration of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, with necitumumab sequentially in any order. As used herein, the phrase “in combination with” also refers to the administration of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or a pharmaceutically acceptable salt thereof, with necitumumab in any combination thereof. 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered prior to administration of necitumumab. 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered at the same time as administration of necitumumab. 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered subsequent to administration of necitumumab. 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered prior to, at the same time as, or subsequent to administration of necitumumab, or in some combination thereof.

Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered prior to each administration of necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered at the same time as each administration of necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered subsequent to each administration of necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered prior to, at the same time as, or subsequent to, each administration of necitumumab or some combination thereof. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered at different intervals in relation to therapy with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a single or series of dose(s) prior to, at any time during, or subsequent to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a single dose prior to, at any time during, or subsequent to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a single dose prior to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a single dose at any time during the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a single dose subsequent to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a series of doses prior to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a series of doses subsequent to the course of treatment with necitumumab. Where necitumumab is administered at repeated intervals (e.g. during a standard course of treatment), 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one can be administered in a series of doses subsequent to the course of treatment with necitumumab.

8-[5-(1-hydroxy-1-methylethyl)pyridin-3-yl]-1-[(2S)-2-methoxypropyl]-3-methyl-1,3-dihydro-2H-imidazo[4,5-c]quinolin-2-one, or pharmaceutically acceptable salts thereof, may be prepared by a variety of procedures known in the art (e.g., see WO 2012/097039).

The following assay results illustrate the unexpected improvement of the combination of necitumumab and 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one in mouse patient-derived xenograft models for squamous histology cancers and certain PI3K pathway activated cancers.

The following assays further illustrate the invention, but should not be construed to limit the scope of the invention in any way. The term “Compound A” refers to 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one which can be made, for example, according to the disclosure in WO 2012/097039. Necitumumab can be made, for example, according to the disclosure in U.S. Pat. No. 7,598,350.

Antitumor Effects of Compound A in Combination with Necitumumab in Mouse Patient-Derived Xenograft Models for Squamous and PI3K Pathway Activated Cancers General Information:

### Tumor Implantation and Treatment

Establish patient-derived xenograft (PDX) tumor models from viable human tumor tissue and serially passage in immunocompromised female mice a limited number of times. Using tumor fragments harvested from donor host animals, perform unilateral subcutaneous implants from a specific passage of the PDX tumor model on the flanks of experimental animals. When mean tumor volumes reach approximately 100-200 mm³ in size, randomize the animals by tumor size and body weight by randomization techniques well known in the art and place into their respective 4 treatment groups using 5 animals per treatment group. Formulate Compound A weekly in 1% hydroxyethyl cellulose (HEC)/0.25% Tween 80 (Polysorbate 80)/0.05% Dow-Corning Antifoam 1510-US and administer by oral gavage once-daily for 28 or 35 days (PO, OD×28-35) at a dose of 30 ma/kg. Formulate necitumumab by dilution from antibody stock solution using phosphate-buffered saline (PBS) and administer intraperitoneally twice weekly (IP, BIW) at a dose of 20 mg/kg for either 4 or 5 weeks. Dose combination treatment groups with both compounds according to the schedule described above for monotherapy, and dose vehicle treatment groups with both vehicles according to the schedules for Compound A and necitumumab, respectively.

Test the combination of the PI3K/mTOR dual inhibitor small molecule Compound A and human monoclonal anti-EGFR antibody necitumumab, as well as their respective monotherapies, in 21 PDX models (Table 1) across multiple tumor types (squamous NSCLC, squamous bladder, squamous head and neck, squamous thyroid, non-squamous lung large cell, squamous anal or non-squamous colorectal cancer) with and without activating mutations in the PI3K pathway. Assess antitumor efficacy of the treatment groups by measuring tumor volume via caliper measurements twice a week during the course of the study. Measure body weight twice weekly during the course of the study as a general indicator of tolerability.

### Data Capture:

Capture tumor size and body weight twice per week. Estimate tumor volume (V) by using the formula: V=(π/6)×L×W² where L=larger of measured diameter and W=smaller perpendicular diameter. Transform the tumor volume data to a log scale to equalize variance across time and treatment groups. Analyze the log volume data with a two-way repeated measures analysis of variance by time and treatment using the MIXED procedures in SAS software (Version 9.3). The correlation model for the repeated measures is Spatial Power. Compare treated groups compared to the control group at each time point. Use the MIXED procedure also separately for each treatment group to calculate adjusted means and standard errors at each time point. Both analyses account for the autocorrelation within each animal and the loss of data that occurred when animals with large tumors were removed from the study early. Plot the adjusted means and standard errors for each treatment group versus time. Calculate relative changes in tumor volume (% ΔT/C) using the tumor volume measurements taken nearest to the last day of dosing with Compound A, whereas the baseline tumor volume is the volume recorded on or just prior to first day of dosing. Calculate % ΔT/C values using the formula % T/C=100×ΔT/ΔC, whereby T=mean tumor volume of the compound treated group, ΔT=mean tumor volume of the compound treated group minus the mean tumor volume on the baseline day, C=mean tumor volume of the control (vehicle) group, and ΔC=mean tumor volume of the control group minus the mean tumor volume on the baseline day. Tumor growth inhibition is observed in those instances where the calculated values for % TIC are less than 100% whereby greater inhibition results in smaller % T/C values. If ΔT was <0, then a tumor regression value was calculated instead of % T/C whereby % Regression=100×ΔT/Tᵢₙᵢₜᵢₐₗ such that Tᵢₙᵢₜᵢₐₗ=the grand mean of the tumor volume for all the treatment groups. Any negative values for % T/C listed are values for % Regression.

### Results:

Necitumumab monotherapy shows efficacy values ranging from 164.4% ΔT/C to −75.9% regression (Table 2), and shows statistical significance versus vehicle group in 10 of 21 models. Compound A monotherapy shows efficacy values ranging from 110.9% ΔT/C to −62.4% regression (Table 2), and shows statistical significance versus vehicle group in 17 of 21 models. Compound A plus necitumumab combination shows efficacy values ranging from 45.1% ΔT/C to −80.9% regression (fable 2), and shows statistical significance versus vehicle group in all 21 models. The combination group demonstrate actual % ΔT/C values lower than either monotherapy (greater efficacy) in 16 of 21 models (Table 2), while in 6 of those 16 models the combination shows statistically significant differences versus either monotherapy (Table 3). Necitumumab monotherapy demonstrates no or minor group mean body weight losses (Table 4) with no apparent treatment-related mortality. Compound A demonstrates maximum group mean body weight loss of 2 to 12 percent (88 to 98 percent minimum group mean body weight relative to starting body weight upon treatment initiation) in 16 tumor models, with 5 tumor models (TH1442, LU1542, LU0330, BL0597 and CR0047) showing mean body weight loss of greater than 15 percent (less than 85 percent minimum group mean body weight) where individual animals receive dosing holidays or are taken off study if body weight loss is sudden or substantial. Combination treatment demonstrates maximum group mean body weight loss of 2 to 13 percent (87 to 98 percent minimum group mean body weight relative to starting body weight upon treatment initiation) in the same 16 tumor models. Four of the 5 remaining tumor models show mean body weight loss of only 8 to 14 percent (86 to 92 percent minimum group mean body weight) in the combination versus the 16 to 24 percent body weight loss for Compound A monotherapy for those models (again with individual animals receiving dosing holidays or being taken off study if body weight loss is sudden or substantial).

In total, the combination of Compound A and necitumumab shows statistically significant efficacy versus vehicle in 21 of 21 models and greater actual efficacy (lower % ΔT/C or % regression values) than either monotherapy in 16 of 21 models. Six of those 16 models are statistically significant versus either monotherapy. Sixteen of 21 models show maximum mean body weight loss of less than 10 percent upon treatment with the combination. Of the remaining 5 models, the most substantial body weight loss (maximum mean body weight loss 26% while on study) is observed in a single model (CR0047—where maximum body weight loss is greater in the combination than in Compound A monotherapy) while body weight loss is between 8% and 14% in the other four models.

TABLE 1
Bask Information for PDX Models Used

| | | | | | Day |
|---|---|---|---|---|---|
| | | | | Day | analyzed |
| | | | | analyzed | (as |
| Tumor | Tumor | PI3K Pathway | Days | (as Rx | post-implant |
| Model | Type | Activation Status | dosed | day) | day) |
| LXFE 1066 | sq NSCLC | PTEN del (IHC neg) | 35 | 35 | 52 |
| LXFE 937 | sq NSCLC | wt PIK3CA/AKT | 28 | 28 | 49 |
| LXFE 397 | sq NSCLC | wt PIK3CA/AKT | 28 | 28 | 40 |
| LXFE 470 | sq NSCLC | E545K PIK3CA | 28 | 37 | 72 |
| LXFE 646 | sq NSCLC | wt PIK3CA/AKT | 28 | 28 | 45 |
| HNXF 1905 | HNSCC | E545K PIK3CA | 28 | 28 | 48 |
| LXFL 625 | Large Cell Lung | E542Q PIK3CA | 28 | 28 | 42 |
| AXF 984 | sq Anal | E17K AKT1 | 28 | 28 | 101 |
| BXF 1228 | sq Bladder | E542K PIK3CA | 28 | 29 | 43 |
| LU0377 | sq NSCLC | PTEN del (IHC neg) | 28 | 27 | 55 |
| CR0047 | CRC | E545K PIK3CA, KRAS G12C | 28 | 28 | 58 |
| LU1143 | sq NSCLC | E542K PIK3CA | 28 | 27 | 52 |
| LU1423 | sq NSCLC | wt PIK3CA/AKT | 28 | 27 | 63 |
| HN0626 | HNSCC | H1047L PIK3CA | 28 | 27 | 48 |
| LU0858 | sq NSCLC | wt PIK3CA/AKT, | 28 | 27 | 43 |
| | | L858R EGFR | | | |
| LU1542 | sq NSCLC | E17K AKT1 | 28 | 27 | 48 |
| LU1429 | sq NSCLC | wt PIK3CA/AKT, PTEN | 28 | 27 | 48 |
| | | IHC neg | | | |
| BL0597 | sq Bladder | H1047R PIK3CA | 28 | 28 | 61 |
| TH1442 | sq Thyroid | E545K PIK3CA | 28 | 25 | 50 |
| LU0330 | sq NSCLC | E542K PIK3CA | 28 | 28 | 71 |
| LU1215 | sq NSCLC | wt PIK3CA/AKT | 28 | 24 | 42 |

Abbreviations:
sq = squamous;
NSCLC = non-small cell lung carcinoma;
HNSCC = head and neck squamous cell carcinoma;
CRC = colorectal cancer;
wt = wild type;
del =deletion;
IHC = Immunohistochemistry;
neg = negative;
Rx = treatment

TABLE 2
Monotherapy and Combination Therapy Efficacy (vs Vehicle).

| | | Compound A | | Necitumumab | | Combo | |
|---|---|---|---|---|---|---|---|
| | Day | delta[T/C] or | p-value for | delta[T/C] or | p-value for | delta[T/C] or | p-value for |
| Tumor | analyzed | Regresssion | Compound A | Regresssion | Necitumumab | Regresssion | Combo |
| Model | (as Rx day) | (%) | vs. Vehicle | (%) | vs. Vehicle | (%) | vs. Vehicle |
| LXFE 1066 | 35 | 19.2 | 0.002 | 21.5 | 0.003 | 7.6 | <.001 |
| LXFE 937 | 28 | 29.5 | 0.006 | −75.9 | <.001 | −54.9 | <.001 |
| LXFE 397 | 28 | 19.4 | <.001 | 164.4 | 0.089 | 24.6 | <.001 |
| LXFE 470 | 37 | 3.7 | <.001 | 10.4 | 0.001 | −80.9 | <.001 |
| LXFE 646 | 28 | 31.3 | 0.126 | 2.1 | 0.002 | −61.8 | <.001 |
| HNXF1905 | 28 | 67.7 | 0.370 | 2.3 | <.001 | −20.5 | <.001 |
| LXFL 625 | 28 | 24.0 | <.001 | 61.2 | 0.192 | 34.9 | 0.01 |
| AXF 984 | 28 | −5.0 | <.001 | 131.2 | 0.305 | −27.1 | <.001 |
| BXF 1228 | 29 | 19.4 | <.001 | 10.5 | <.001 | 1.3 | <.001 |
| LU0377 | 27 | 42.8 | <.001 | 87.2 | 0.409 | 28.1 | <.001 |
| CR0047 | 28 | 7.9 | <.001 | 43 | 0.001 | −4.8 | <.001 |
| LU1143 | 27 | 26.3 | <.001 | 34.5 | <.001 | 1.7 | <.001 |
| LU1423 | 27 | 44.2 | 0.003 | 84.2 | 0.467 | 28.2 | <.001 |
| HN0626 | 27 | 71.0 | 0.125 | 52.9 | 0.008 | 23.6 | <.001 |
| LU0858 | 27 | 48.9 | 0.027 | 79.9 | 0.417 | 10.1 | <.001 |
| LU1542 | 27 | 34.2 | <.001 | 89.1 | 0.594 | 30.4 | <.001 |
| LU1429 | 27 | 24.9 | <.001 | 88.5 | 0.522 | 15.5 | <.001 |
| BL0597 | 28 | −62.4 | <.001 | 16.5 | <.001 | −49.9 | <.001 |
| TH1442 | 25 | 110.9 | 0.923 | 43.7 | 0.225 | −69.9 | <.001 |
| LU0330 | 28 | −41.2 | <.001 | 26.4 | 0.092 | −64.0 | <.001 |
| LU1215 | 24 | 43.1 | 0.021 | 68.1 | 0.250 | 45.1 | 0.023 |

Abbreviations:
Rx = treatment;
Combo = Combination

TABLE 3
Combination p-value Assessment per Model

| | Day | Combo | p-value for | p-value for | p-value for |
|---|---|---|---|---|---|
| Tumor | analyzed | delta[T/C] or | Combo vs. | Compound A | Necitumumab |
| Model | (as Rx day) | Regresssion (%) | Vehicle | vs. Combo | vs Combo |
| LXFE 1066 | 35 | 7.6 | <.001 | 0.186 | 0.131 |
| LXFE 937 | 28 | −54.9 | <.001 | <.001 | 0.043 |
| LXFE 397 | 28 | 24.6 | <.001 | 0.464 | <.001 |
| LXFE 470 | 37 | −80.9 | <.001 | <.001 | <.001 |
| LXFE 646 | 28 | −61.8 | <.001 | <.001 | 0.08 |
| HNXF 1905 | 28 | −20.5 | <.001 | <.001 | 0.34 |
| LXFL 625 | 28 | 34.9 | 0.01 | 0.348 | 0.13 |
| AXF 984 | 28 | −27.1 | <.001 | 0.218 | <.001 |
| BXF 1228 | 29 | 1.3 | <.001 | <.001 | 0.036 |
| LU0377 | 27 | 28.1 | <.001 | 0.06 | <.001 |
| CR0047 | 28 | −4.8 | <.001 | 0.156 | <.001 |
| LU1143 | 27 | 1.7 | <.001 | <.001 | <.001 |
| LU1423 | 27 | 28.2 | <.001 | 0.151 | <.001 |
| HN0626 | 27 | 23.6 | <.001 | <.001 | 0.008 |
| LU0858 | 27 | 10.1 | <.001 | <.001 | <.001 |
| LU1542 | 27 | 30.4 | <.001 | 0.64 | <.001 |
| LU1429 | 27 | 15.5 | <.001 | 0.09 | <.001 |
| BL0597 | 28 | −49.9 | <.001 | 0.417 | <.001 |
| TH1442 | 25 | −69.9 | <.001 | <.001 | <.001 |
| LU0330 | 28 | −64.0 | <.001 | 0.175 | <.001 |
| LU1215 | 24 | 45.1 | 0.023 | 0.97 | 0.212 |

Abbreviations:
Rx = treatment;
Combo = Combination

TABLE 4
Minimum Mean Body Weight Reached During Treatment in Each Group
Across 21 Tumor Models as a Percentage of Initial Body Weight Upon
Treatment Initiation

| | Minimum Group Mean Body Weight on study (as % of group | | | |
|---|---|---|---|---|
| | mean body weight upon treatment initiation) | | | |
| | | | | Compound A + |
| Tumor | | | | Necitumumab |
| Model | Vehicle | Compound A | Necitumumab | Combination |
| LXFE 1066 | 98.8 | 92.1 | 96.6 | 95.3 |
| LXFE 937 | 100.0 | 96.9 | 99.5 | 96.1 |
| LXFE 397 | 97.9 | 96.9 | 97.6 | 92.6 |
| LXFE 470 | 94.7 | 88.9 | 96.6 | 90.7 |
| LXFE 646 | 100.0 | 88.6 | 100.0 | 87.5 |
| HNXF 1905 | 96.6 | 98.6 | 99.3 | 96.9 |
| LXFL 625 | 100.0 | 95.3 | 99.4 | 94.2 |
| AXE 984 | 100.0 | 97.4 | 99.4 | 96.5 |
| BXF 1228 | 99.4 | 96.6 | 96.7 | 94.2 |
| LU0377 | 99.1 | 94.6 | 99.5 | 97.0 |
| CR0047 | 95.0 | 82.8 | 98.7 | 74.2 |
| LU1143 | 100.0 | 96.5 | 100.0 | 97.4 |
| LU1423 | 96.0 | 89.3 | 100.0 | 91.1 |
| HN0626 | 99.8 | 97.6 | 99.5 | 92.3 |
| LU0858 | 96.7 | 91.5 | 100.0 | 89.1 |
| LU1542 | 100.0 | 83.4 | 100.0 | 90.1 |
| LU1429 | 100.0 | 98.5 | 100.0 | 99.2 |
| BL0597 | 99.8 | 76.5 | 99.7 | 86.9 |
| TH1442 | 100.0 | 83.6 | 100.0 | 91.2 |
| LU0330 | 97.1 | 80.4 | 91.9 | 89.7 |
| LU1215 | 100.0 | 89.5 | 100.0 | 98.4 |

A Study of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one in combination with necitumumab for patients with histologically-confirmed advanced or metastatic squamous non-small cell lung cancers (NSCLC), with progressive disease after receiving a platinum-based chemotherapy regimen.

### Study Design

The study is a Phase II single-arm, open-label, clinical study of the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab in patients with previously treated advanced or metastatic squamous non-small cell carcinoma of the lung.

### Study Objectives

The primary objective of this study is to evaluate the 6-month disease control rate (DCR) in patients receiving the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab after first-line platinum-based chemotherapy regimen for advanced or metastatic squamous non-small cell carcinoma of the lung.

The secondary objectives of this study are 1) to establish that the doses of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab being studied are safe and well-tolerated when administered in combination, 2) to characterize exposure of necitumumab and 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one when administered in combination, and 3) to evaluate additional measures of efficacy including overall response rate (ORR), progression-free survival (PFS), and overall survival (OS) of the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab after first-line platinum-based chemotherapy for advanced or metastatic squamous non-small cell carcinoma of the lung. The exploratory objective of this study is to potentially identify biomarkers (including but not limited to biomarkers of the EGFR and PI3K/mTOR pathways) associated with clinical efficacy and disease progression in this patient population.

### Treatment Plan

8-[5-(1-hydroxy-1-methylethyl)pyridin-3-yl]-1-[(2S)-2-methoxypropyl]-3-ethyl-1,3-dihydro-2H-imidazo[4,5-c]quinolin-2-one (200 mg orally BID) and necitumumab (800 mg administered IV on Day 1 and 8 of each 21-day cycle) Cycles will be 21 days in length. Patients will be treated until disease progression as defined by RECIST v1.1, or any other discontinuation criteria outlined in below as applicable. Patients will be evaluated for response to treatment after every 2 cycles.

### Safety Lead-In Cohort

Since the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab will be given for the first time to humans, a safety lead-in will be conducted. After at least 6 patients (lead-in cohort) have been treated for a full cycle, a Safety Internal Monitoring Committee (SIMC) will conduct a review of the safety and available PK data to evaluate the safety and PKs of the combination. If 2 (or more) of 3 or 2 (or more) of 6 patients in the lead-in cohort experience dose-limiting toxicities (DLTs) as defined below, 3 to 6 additional patients will be treated at a lower dose of study drug(s) following discussion of the safety data by the SIMC and assessed for DLTs. If there is a safety concern or PK interaction deemed to be clinically significant by the SIMC, the SIMC may recommend enrollment of approximately 6 additional patients to further evaluate the safety of the combination, or explore other doses of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one in combination with necitumumab. In the case of unacceptable and/or unmanageable toxicity of the combination at the intended dose level, the SIMC may decide to discontinue or modify the study (e.g. proceed with a lower dose level of study drug(s) tolerated in combination).

### Post Lead-In Cohort

Following the confirmation of safety for the combination of 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab by the SIMC, the study will continue to enroll up to a total of approximately 48 evaluable patients, with a planned interim analysis after 24 patients have completed 6 months of follow-up. The interim analysis will be performed purely for the purpose of detecting an efficacy signal, and not for the purpose of stopping recruitment. Cycles will be 21 days in length. Patients will be treated until disease progression as defined by RECIST v1.1, or any other discontinuation criteria outlined in below as applicable. Patients will be evaluated for response to treatment after every 2 cycles. As further clinical data become available, this clinical trial may be amended to include the addition of other agents to the combination of necitumumab and/or 8-[5-(1-hydroxy-1-methylethyl)pyridin-3-yr]-1-[(2S)-2-methoxypropyl]-3-methyl-1,3-dihydro-2H-imidazo[4,5-c]quinolin-2-one.

### Response Assessment

Patients will be evaluated radiographically for response to treatment prior to Cycle 3 and after every 2 cycles of treatment (i.e. Cycle 5, Cycle 7, etc.). The following assessments will be performed:
CT scans of chest
CT of the abdomen and pelvis
CT/MRI of the brain only if abnormal at baseline

Patients with progressive disease (PD) or unacceptable toxicity should be discontinued from the study; patients with stable disease (SD) or response to therapy will continue treatment.

### Dose Limiting Toxicity

A toxicity will be considered dose-limiting, if it occurs within the first cycle of treatment (21 days) to lead-in patients and is deemed at least possibly related to study drug. Dose-limiting toxicities (DLTs) will be defined as any of the following adverse events (AEs): Grade 4 thrombocytopenia; Grade 4 neutropenia ≥7 days; Grade ≥3 febrile neutropenia;

and Grade≥3 thrombocytopenia with Grade≥2 hemorrhage Grade≥3 non-hematologic toxicity despite maximal medical management with the exception of:
Diarrhea, nausea, or vomiting that resolves to ≤Grade 2 within 48 hours
Skin toxicity that resolves to ≤Grade 2 within 7 days
Alanine aminotransferase (ALT) and aspartate aminotransferase (AST) elevation that resolves to ≤Grade 2 within 7 days
Hypomagnesemia that resolves to ≤Grade 2 within 7 days
Grade 3 mucositis that resolves to ≤Grade 2 within 7 days. Grade 4 mucositis of any duration will be considered a DLT.
Grade 3 fasting hyperglycemia that resolves to ≤Grade 2 within 7 days. Grade 4 hyperglycemia of any duration that results in intensive care unit admission will be considered a DLT,
Grade 3 fatigue that resolves to ≤Grade 2 within 5 days.
Grade 3 hypertension controlled with medical therapy.
Any lab abnormalities that are not clinically significant and resolve in 72 hours.
Infusion reactions attributed only to necitumumab
Hy's law: Hepatocellular injury defined as ALT (or AST) >3× upper limit of normal (ULN) and total bilirubin >2×ULN with no significant cholestasis (alkaline phosphatase <2×ULN) and no other cause which explains the abnormality in liver tests.
Grade >2 skin toxicities despite best supportive care
Unresolved NE that leads to treatment delay of ≥14 days
Any other clinically significant study drug related AE which does not respond to supportive care, or is judged to be an unacceptable and/or unmanageable by the investigator in collaboration with the Medical Monitor.

### Determination of Dose-Limiting Toxicities

The patient population used for determination of DLTs will consist of patients who have met the minimum safety evaluation requirements of the study, and/or who have experienced a DLT. Minimum safety requirements will be met if, during Cycle 1 of treatment, the patient receives at least 75% of study drug regimen (8-[5-(1-hydroxy-1-methylethyl)pyridin-3-yl]-1-[(2S)-2-methoxypropyl]-3-methyl-1,3-dihydro-2H-imidazo[4,5-c]quinolin-2-one and necitumumab) and is observed for at least 21 days following the first dose of study drugs. Any patients enrolled in the lead-in cohort that do not meet the dosing criteria defined above in order to be considered complete will be replaced.

Discontinuation from Study Treatment

Patients will be discontinued from study treatment for any of the following reasons:
Disease progression
Irreversible or intolerable AE thought to be related to study drug(s) (e.g. infusion related reactions ≥Grade 3 to necitumumab)
Conditions requiring therapeutic intervention not permitted by the protocol Intercurrent illness (this will be at the investigator's discretion)
Inability of the patient to comply with study requirements or lost to follow-up
Patient requests to discontinue treatment
Patient withdraws consent
Investigator or Sponsor team, for any ethical, medical, or scientific reason, while considering the rights, safety, and well-being of the patient(s), stops the study treatment.
Any patient requiring an AE-related dose delay of more than 21 days due to a study drug related AE must be discontinued from the study treatment, unless discussed with the Medical Monitor.

We claim:

1. A method of treating squamous histology cancers and PI3K pathway activated large cell lung and colorectal cancers in a patient, comprising administering to a patient in need of such treatment an effective amount of a compound of the formula: 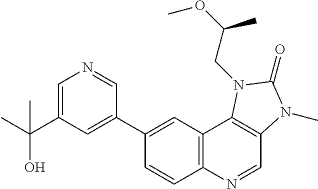 [SMILES: CO[C@@H](C)Cn1c(=O)n(C)c2cnc3ccc(-c4cncc(C(C)(C)O)c4)cc3c21]
or a pharmaceutically acceptable salt thereof, in combination with an effective amount of necitumumab.

2. The method of claim 1, wherein the compound is

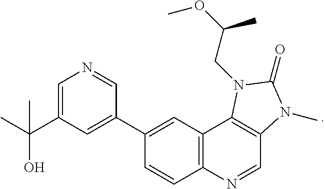 [SMILES: CO[C@@H](C)Cn1c(=O)n(C)c2cnc3ccc(-c4cncc(C(C)(C)O)c4)cc3c21]

3. The method of claim 1, wherein the squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC.

4. The method of claim 1, wherein the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC.

5. The method of claim 1 wherein the squamous histology cancers and PI3K pathway activated large cell lung and colorectal cancer is squamous NSCLC.

6. The method of claim 1 wherein the compound or salt thereof is administered at a dose of about 200 mg twice per day of a 21-day cycle and necitumumab is administered at a dose of about 800 mg on Days 1 and 8 of a 21-day cycle.

7. The method of claim 6 wherein the compound or salt thereof is administered orally and necitumumab is administered intravenously.

8. A kit comprising a compound of the formula: 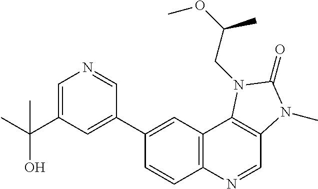 [SMILES: CO[C@@H](C)Cn1c(=O)n(C)c2cnc3ccc(-c4cncc(C(C)(C)O)c4)cc3c21]
or a pharmaceutically acceptable salt thereof, and necitumumab for the treatment of squamous histology cancers and PI3K pathway activated large cell lung and colorectal cancers.

9. The kit of claim 8 wherein the compound is

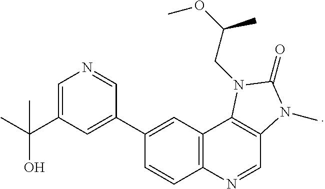 [SMILES: CO[C@@H](C)Cn1c(=O)n(C)c2cnc3ccc(-c4cncc(C(C)(C)O)c4)cc3c21]

10. The kit of claim 8, wherein the squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC.

11. The kit of claim 8, wherein the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC.

12. The kit of claim 8 wherein the squamous histology cancers and PI3K pathway activated large cell lung and colorectal cancer is squamous NSCLC.

13. A kit, comprising a pharmaceutical composition, comprising a compound of the formula: 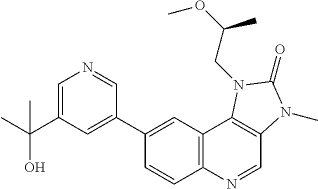 [SMILES: CO[C@@H](C)Cn1c(=O)n(C)c2cnc3ccc(-c4cncc(C(C)(C)O)c4)cc3c21]
or a pharmaceutically acceptable salt thereof, with one or more pharmaceutically acceptable carriers, diluents, or excipients, and a pharmaceutical composition, comprising necitumumab, with one or more pharmaceutically acceptable carriers, diluents, or excipients for the treatment of squamous histology cancers and PI3K pathway activated large cell lung and colorectal cancers.

14. The kit of claim 13, wherein the squamous histology cancers and PI3K pathway activated large cell lung and colorectal cancers are squamous NSCLC, HNSCC, squamous anal cancer, squamous bladder cancer, squamous thyroid cancer, non-squamous large cell lung cancer, and non-squamous CRC.

15. The kit of claim 13, wherein the squamous histology cancers and PI3K pathway activated large cell lung and colorectal cancers are HNSCC, squamous anal cancer, squamous bladder cancer, squamous thyroid cancer, non-squamous large cell lung cancer, and non-squamous CRC.

16. The kit of claim 13 wherein the squamous histology cancers and PI3K pathway activated large cell lung and colorectal cancer is squamous NSCLC.

17-24. (canceled)


SEQUENCE LISTING (2 sequences)

SEQ ID NO: 1 - protein, 121 aa, Homo sapiens [sequence omitted]

SEQ ID NO: 2 - protein, 107 aa, Homo sapiens [sequence omitted]